(19) United States
(12) Patent Application Publication
(10) Pub. No.: US 2012/0165651 A1
(43) Pub. Date: Jun. 28, 2012

(54) DETECTOR ROTATION TYPE RADIATION THERAPY AND IMAGING HYBRID DEVICE

(75) Inventors: Taiga Yamaya, Chiba-shi (JP); Eiji Yoshida, Chiba-shi (JP); Fumihiko Nishikido, Chiba-shi (JP); Taku Inaniwa, Chiba-shi (JP); Hideo Murayama, Chiba-shi (JP)

(73) Assignee: NATIONAL INSTITUTE OF RADIOLOGICAL SCIENCES, Chiba-shi, Chiba (JP)

(21) Appl. No.: 13/257,526

(22) PCT Filed: Mar. 23, 2009
(86) PCT No.: PCT/JP2009/055701

§ 371 Date: Mar. 1, 2012

(51) Int. Cl. A61B 6/00

(52) U.S. Cl. 600/411

(57) ABSTRACT

An imaging device, or a PET device, opposed gamma camera type PET device, or open PET device in particular, that is combined with a radiation therapy device, in which detectors are rotated to reduce incidence of nuclear fragments on the detectors. For example, in an opposed gamma camera type PET device, beam irradiation and detector rotation can be synchronized to prevent the detectors from interfering with the treatment beam and reduce the incidence of nuclear fragments on the detectors. This makes it possible to reduce the incidence of nuclear fragments on the detectors without interfering with a treatment beam, thereby enabling measurement of annihilation radiations and three-dimensional imaging of the irradiation field immediately after irradiation or during irradiation.

### TECHNICAL FIELD

The present invention relates to a radiation therapy and imaging hybrid device that can reduce, when performing monitoring for detecting annihilation radiations occurring from an irradiation field due to radiation (also referred to as beam) irradiation in radiation therapy which is conducted by irradiating an affected area with X-rays or a particle beam, the incidence of nuclear fragments on detectors without interfering with the treatment beam, thereby enabling measurement of annihilation radiations and three-dimensional imaging of the irradiation field immediately after irradiation or during irradiation.

### BACKGROUND ART

Positron emission tomography (PET) is attracting attention as an effective test method for earlier diagnosis of cancer. In PET, a compound labeled with a trace amount of positron emitting nuclei is administered and annihilation radiations emitted from inside the body are detected to create an image of metabolic functions such as sugar metabolism and check for a disease and its extent. PET devices for practicing it have been put into actual use.

The principle of PET will be described below. A positron emitted from a positron emitting nuclide due to positron decay is annihilated with an ambient electron to produce a pair of 511-keV annihilation radiations, which are measured by a pair of radiation detectors based on the principle of coincidence counting. The position of the nuclide can thus be located on a single line (line of coincidence) that connects the pair of detectors. An axis from the patient's head to feet will be defined as a body axis. The distribution of nuclei on a plane that perpendicularly crosses the body axis is determined by two-dimensional image reconstruction from data on lines of coincidence on the plane, measured in various directions.

Early PET devices therefore have had a single-ring detector in which detectors are closely arranged in a ring shape on a plane to be the field of view so as to surround the field of view. With the advent of a multi-ring detector which includes a lot of single-ring detectors closely arranged in the direction of the body axis, the two-dimensional field of view has subsequently been extended to three dimensions. Since the 1990s, 3D mode PET devices have been actively developed which perform coincidence measurement even between detector rings with a significant improvement in sensitivity.

For cancer detected by the PET diagnosis or the like, treatments have a critical role. Approaches other than surgical operations and medication include radiation therapy of irradiating the affected area with radiations such as X-rays and gamma rays. In particular, particle radiotherapy of irradiating only a cancerous area with a heavy particle beam or proton beam is attracting much attention as a method both with an excellent treatment effect and a sharply concentrated irradiation characteristic with respect to the affected area. Among the methods of particle beam irradiation is conventional bolus irradiation where the irradiating beam is spread out to the shape of the affected area. In addition, spot scanning irradiation is under study, where the affected area is scanned with a pencil beam according to its shape etc. In any case, the directions and dosages of the irradiation beam are precisely controlled according to a treatment plan that is thoroughly calculated based on X-ray CT images or the like obtained separately.

The patient positioning accuracy is the key to administer treatment exactly according to the treatment plan. The irradiation field is often positioned based on X-ray images. In general, X-ray images fail to provide a sufficient contrast between tumor and normal tissues, and it is difficult to identify a tumor itself for positioning. In addition to such misalignment of the irradiation field at the time of patient setup, other problems have been pointed out such as a change in the size of the tumor from the time of creation of the treatment plan, and respiratory and other variations of the tumor position. Under the present circumstances, it is difficult to accurately identify whether irradiation is performed according to the treatment plan. Even if the actual irradiation field deviates from the treatment plan, it is not easy to detect.

To solve the foregoing problems, attention is being given to a method of imaging the irradiation field in real time using PET techniques. In the method, no PET medicine is administered. Instead, annihilation radiations caused by particle beam irradiation or X-ray irradiation through a projectile fragmentation reaction, target fragmentation reaction, and photonuclear reaction are rendered into an image by using the principle of PET. Therapy monitoring is said to be possible since the positions of occurrence of annihilation radiations has a strong correlation with the dose distribution of the irradiation beam. (W. Enghardt, et al. “Charged hadron tumour therapy monitoring by means of PET,” Nucl. Instrum. Methods A 525, pp. 284-288, 2004. S. Janek, et al. “Development of dose delivery verification by PET imaging of photonuclear reactions following high energy photon therapy,” Phys. Med. Biol., vol. 51 (2006) pp. 5769-5783). In heavy particle radiotherapy, direct irradiation of positron-emitting nuclei such as ¹¹C, instead of ordinary stable nuclei such as ¹²C, can eliminate a mismatch between the positions of occurrence of annihilation radiations and the dose distribution, as well as improve the S/N ratio of PET images.

Device requirements for PET that images an irradiation field in real time (hereinafter, referred to as beam on-line

PET) are summarized into the following four points:

1. The detectors not obstruct the treatment beam.

2. The detectors not drop in performance due to nuclear fragments (charged particles and/or neutrons generated by collision of incident particles and target nuclei).

3. PET measurement can be performed immediately after irradiation or even during irradiation for efficient measurement of short life RIs, in order to enhance the precision of PET images and shorten the patient binding time.

4. The irradiation field can be imaged in a three-dimensional fashion.

Concerning the foregoing requirement 2, the incident of nuclear fragments on detectors can radioactivate the scintillators themselves that constitute the detectors. This may lead to the omission of annihilation radiations to be measured and the production of errors in position information. In heavy particle beam irradiation, both charged particles and neutrons occur as nuclear fragments. In proton beam irradiation, neutrons are considered to be dominant. In either case, nuclear fragments are generated with forward directivity with respect to the treatment beam. It has been reported that the forward directivity is accompanied by a wide angle. (N. Matsufuji, et al., “Spatial fragmentation distribution from a therapeutic pencil-like carbon beam in water,” Physics in Medicine and Biology 50(2005) 3393-3403, S. Yonai, et al., “Measurement of neutron ambient dose equivalent in passive carbon-ion and proton radiotherapies,” Medical Physics 35 (2008) 4782-4792).

As for the requirement 3, the nuclei generated by the radiation irradiation have an extremely short half-life of several tens of seconds to 20 minutes or so. The nuclei can also move inside the living body due to blood flow and other factors. Immediate PET measurement during irradiation is thus desired.

The GSI Laboratory in Germany and the National Cancer Center Hospital East in Japan are making attempts for beam on-line PET by using an opposed gamma camera type PET device in which two PET detectors of flat type are arranged with the bed of a treatment device therebetween. (P. Crespo, et al., “On the detector arrangement for in-beam PET for hadron therapy monitoring,” Phys. Med. Biol., vol. 51 (2006) pp. 2143-2163, T. Nishio, et al., “Dose-volume delivery guided proton therapy using beam ON-LINE PET system,” Med. Phys., vol. 33 (2006) pp. 4190-4197). The opposed gamma camera type device satisfies the requirements 1, 2, and 3 since the detectors can be arranged away from the beam path. However, lines of coincidence measured are highly uneven in direction, some information necessary for image reconstruction missing. This significantly reduces the resolution in directions perpendicular to the detector plane, failing to meet the requirement 4.

To satisfy the requirement 4, lines of coincidence from various directions need to be measured. In terms of a PET device itself, there has been proposed a device that is configured to rotate its opposed gamma camera type device (David Townsend, et al., “A Rotating PET Camera using BGO Block Detectors,” Conference Record of the 1991 IEEE Nuclear Science Symposium and Medical Imaging Conference). When combined with a radiotherapy device, such a PET device in turn fails to meet the requirements 1 and 2 because of interference between the PET detectors and the treatment beam.

There have also been proposed methods of implementing opposed gamma camera type PET device on a rotating treatment gantry in which the treatment beam irradiation device itself rotates around a patient (Japanese Patent Application Laid-Open No. 2008-22994, Japanese Patent Application Laid-Open No. 2008-173299). The requirement 3 is not satisfied, however, since the opposed gamma camera type PET device can be rotated only after beam irradiation, except in some rare cases where beam irradiation is continuously performed from various directions.

The applicant has proposed an open PET device as a method that provides a gap for allowing the passage of a treatment beam and is capable of three-dimensional imaging without rotation of the PET device. In the open PET device, as shown in FIG. 1, two separate multi-ring detectors 22 and 24 are arranged apart in the direction of the body axis of a patient 8 to form a physically open field of view area (also referred to as an open field of view) (Taiga Yamaya, Taku Inaniwa, Shinichi Minohara, Eiji Yoshida, Naoko Inadama, Fumihiko Nishikido, Kengo Shibuya, Chih Fung Lam and Hideo Murayama, “A proposal of an open PET geometry,” Phy. Med. Biol., 53, pp. 757-773, 2008.). Images in the open field of view are reconstructed from lines of coincides between the two separate detector rings 22 and 24. In the diagram, 10 designates a bed, 12 designates a pedestal of the bed, and 26 designates a gantry cover. Such an open PET device meets the requirements 1, 3, and 4. Without an open field of view of sufficient width, nuclear fragments 34 caused by a treatment beam 32 that enters the open field of view from an irradiation port 30 may be incident on detectors on both sides of the open field of view. If the treatment intensity is extremely high, the detectors may get radioactivated and fail to meet the requirement 2.

### SUMMARY OF THE INVENTION

The present invention has been achieved in order to solve the foregoing conventional problems. It is an object of the present invention to reduce the incidence of nuclear fragments on detectors without interfering with a treatment beam, thereby enabling measurement of annihilation radiations and three-dimensional imaging of the irradiation field immediately after irradiation or during irradiation.

The present invention is directed to an imaging device, or a PET device, opposed gamma camera type PET device, or open PET device in particular, that is combined with a radiation therapy device, in which detectors are rotated to reduce the incidence of nuclear fragments on the detectors.

For example, in an opposed gamma camera type PET device, beam irradiation and detector rotation can be synchronized to prevent the detectors from interfering with the treatment beam and reduce the incidence of nuclear fragments on the detectors.

The present invention has been achieved in view of the foregoing findings, and solves the foregoing object by the provision of a radiation therapy and imaging hybrid device including an imaging device that has a detector arranged so as to be able to measure a secondary radiation occurring from an affected area due to radiation irradiation and images an irradiation field after irradiation or during irradiation in synchronization with a radiation with which a field of view of the detector is irradiated, the hybrid device including: a radiation therapy device that irradiates a part of a subject with a radiation from a predetermined direction, the part lying in a field of view of the imaging device; the detector that is arranged so as to be rotatable about the field of view; and means for controlling rotation of the detector so as to lessen incidence of nuclear fragments on the detector, the nuclear fragments flying forward in a direction of irradiation from the subject due to the radiation irradiation.

Here, the detector may be a group of detectors that are opposed to and paired with each other with the subject interposed therebetween so as to be capable of coincidence measurement of a pair of annihilation radiations occurring from the subject. The imaging apparatus may be a PET device that performs tomographic scanning on the subject.

The irradiation of the radiation may be performed in a region where nuclear fragments are not incident on the detector, and the irradiation of the radiation may be stopped when the detector approaches a region where nuclear fragments are incident, the regions lying on an orbit of rotation of the detector.

The group of detectors may be formed in a discontinuous ring shape. A radiation irradiation path for irradiating the subject with the radiation may be arranged to pass the discontinuous ring. Rotation of the group of detectors may be controlled to locate a discontinuous part of the ring to a position across the radiation irradiation path during radiation irradiation so that the subject is irradiated with the radiation through the discontinuous part.

There may be arranged a plurality of the discontinuous part. The discontinuous part to be located across the radiation irradiation path may be switched based on a predetermined plan when radiation irradiation is at rest.

The group of detectors may be formed in a ring shape about an axis of the subject. Two of the ring-shaped detector may be opposed to each other with a gap therebetween. A radiation irradiation path for irradiating the subject with the irradiation may be arranged in the gap. A detector on the ring of the ring-shaped detector may be missing.

Opposing two detectors on the ring of the ring-shaped detector may be missing.

The detector may be formed in a ring shape. The ring-shaped detector may make continuous rotations during radiation irradiation to distribute a degree of radioactivation over respective detectors.

When the radiation irradiation is periodically performed, a period of rotation of the ring-shaped detector may be other than an integer multiple of a period of irradiation of the radiation.

A degree of radioactivation of the detector radioactivated by the nuclear fragments may be detected, and if the degree of radioactivation of a detected part is detected to reach or exceed a predetermined value, the ring-shaped detector may be rotated and retracted by a predetermined angle to a position of less radioactivation.

The predetermined angle may be an angle set in advance.

The predetermined angle may be an angle by which the ring-shaped detector is rotated after the detecting means detects that the degree of radioactivation of the detected part reaches or exceeds a first predetermined value, until a level of radioactivation detected by the detecting means falls to or below a second predetermined value that is a level lower than or equal to the first predetermined value.

Two of the ring-shaped detector may be opposed to each other with a gap therebetween. A radiation irradiation path for irradiating the subject with the radiation may be arranged in the gap.

An axis of the ring-shaped detector(s) may be oblique to an axis of the subject.

The group of detectors may be opposed to beside the subject.

The degree of radioactivation of the detector radioactivated by the nuclear fragments may be detected, and if the group of detectors formed in the ring shape has a plurality of discontinuous parts and the degree of radioactivation of the detected part is detected to reach or exceed a predetermined value, the discontinuous part to be located across the radiation irradiation path may be switched when the radiation irradiation is at rest.

The degree of radioactivation may be detected from a measured value per unit time, the measured value being calculated for each element of the detector.

The group of detectors may make a swing movement.

A swing angle may be smaller than or equal to 360°.

The present invention also provides a control program of a detector rotation type radiation therapy and imaging hybrid device including an imaging device that has a detector arranged so as to be rotatable about a subject and so as to be able to measure a radiation occurring from an affected area due to radiation irradiation and images an irradiation field after irradiation or during irradiation in synchronization with a radiation with which a field of view of the detector is irradiated, the control program controlling rotation of the detector so as to lessen incidence of nuclear fragments on the detector, the nuclear fragments flying forward in a direction of irradiation from the subject due to the radiation irradiation.

Here, the detector may be a group of detectors that are opposed to and paired with each other with the subject interposed therebetween so as to be capable of coincidence measurement of a pair of annihilation radiations occurring from the subject. The imaging apparatus may be a PET device that performs tomographic scanning on the subject.

If a degree of radioactivation of the detector radioactivated by the nuclear fragments is detected to reach or exceed a predetermined value, the group of detectors may be rotated by an angle set in advance.

If a degree of radioactivation of the detector radioactivated by the nuclear fragments is detected to reach or exceed a first predetermined value, a ring-shaped detector may be rotated until a level of radioactivation detected falls to or below a second predetermined value that is a level lower than or equal to the first predetermined value.

The group of detectors may make a swing movement with a swing angle of 360° or less.

According to the present invention, when performing monitoring for detecting annihilation radiations occurring from an irradiation field due to radiation irradiation in radiation therapy which is conducted by irradiating an affected area with X-rays or a particle beam, it is possible to reduce the incidence of nuclear fragments on detectors without interfering with the treatment beam, thereby enabling measurement of annihilation radiations and three-dimensional imaging of the irradiation field immediately after irradiation or even during irradiation.

### BRIEF DESCRIPTION OF THE DRAWINGS

FIG. 1 includes a front view and a side view showing an open PET device proposed by the applicant;

FIG. 2 is a side view showing a conventional problem;

FIG. 3 is a side view showing an embodiment of the present invention;

FIG. 4 is a block diagram showing a configuration for synchronizing beam irradiation with detector rotation according to the above embodiment;

FIG. 5 is a flowchart showing a typical procedure of beam irradiation in synchronization with detector rotation according to the present invention;

FIG. 6 is a flowchart showing a modification of the procedure of FIG. 5;

FIG. 7 is a time chart showing how beam irradiation and detector rotation are synchronized to prevent the detectors from interfering with a treatment beam or being affected by nuclear fragments according to the embodiment;

FIG. 8 is a side view showing another embodiment of the present invention;

FIG. 9 is a longitudinal sectional view taken from the front, showing a practical example of the detector rotation type radiation therapy and PET hybrid device according to the present invention;

FIG. 10 is a cross-sectional view near the center of FIG. 9;

FIG. 11 is a perspective view showing essential parts of an example where the PET detector rotation according to the present invention is applied to an open PET device;

FIG. 12 is a plan view showing an example in which a PET detector ring is obliquely arranged;

FIG. 13 is a perspective view showing an example where the present invention is applied to an opposed gamma camera type PET device;

FIG. 14 is a flowchart showing a typical procedure for sensing the degree of radioactivation and rotating the detectors to distribute the incidence of fragments for reduced detector damage according to the present invention;

FIG. 15 is a perspective view showing essential parts of another example where the method of rotation type PET according to the present invention is applied to an open PET device;

FIG. 16 is a perspective view showing a configuration in which unnecessary gaps are filled with detectors to improve the sensitivity of PET measurement;

FIG. 17 is a side view of FIG. 16; and

FIG. 18 is a time chart showing how beam irradiation and detector rotation are synchronized to avoid the incidence of nuclear fragments on detectors according to the present invention.

### BEST MODE CARRYING OUT THE INVENTION

Hereinafter, embodiments of the present invention will be described in detail with reference to the drawings.

In an embodiment of the present invention, two detectors are installed with a bed therebetween. The detectors have the function of rotating about the bed independent of an irradiation port. The detectors here have an arc-shaped configuration, whereas they may have a flat configuration. The irradiation port here is a fixed irradiation port, whereas it may be a rotating treatment gantry.

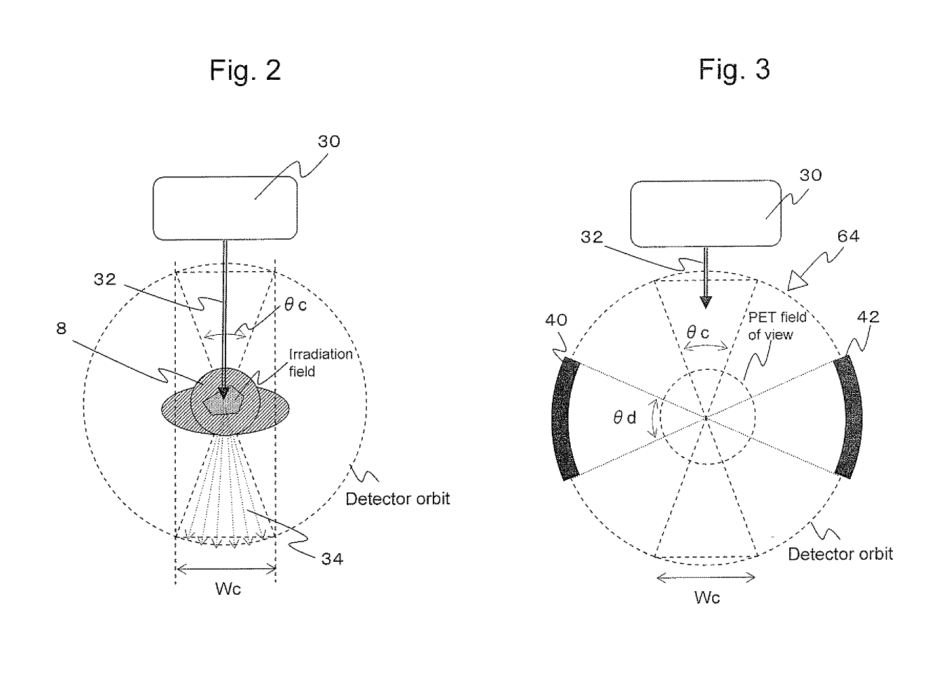

FIG. 2 shows a situation where detectors can interfere with a treatment beam 32 or can be affected by nuclear fragments 34. Wc represents the range not to be obstructed by any detector during beam irradiation (hereinafter, referred to as a critical region). The relationship between the angle of view θc from the center of rotation of the detectors and Wc is expressed as θc=2 sin⁻¹(Wc/(2R)). R is the radius of the orbit of the detectors. Among the methods of particle beam irradiation is conventional bolus irradiation where an affected area is irradiated with a beam that is spread out according to the shape of the affected area. In addition, spot scanning irradiation is under study, where the affected area is scanned with a pencil beam according to its shape etc. In any case, the width of the treatment beam itself is on the order of the maximum width of the irradiation field. In fact, nuclear fragments 34 occurring from range shifters (not shown) or the like in the irradiation port 30 or occurring from inside the body of the patient 8 are considered to spread out wider. Wc or θc is thus defined as the range of effect of the nuclear fragments 34 on the detector orbit.

FIG. 3 shows the configuration of the present embodiment. The present embodiment has the structure that a pair of arc-shaped detectors 40 and 42 are opposed to each other, with an angle of view θd when seen from the center of rotation of the detectors.

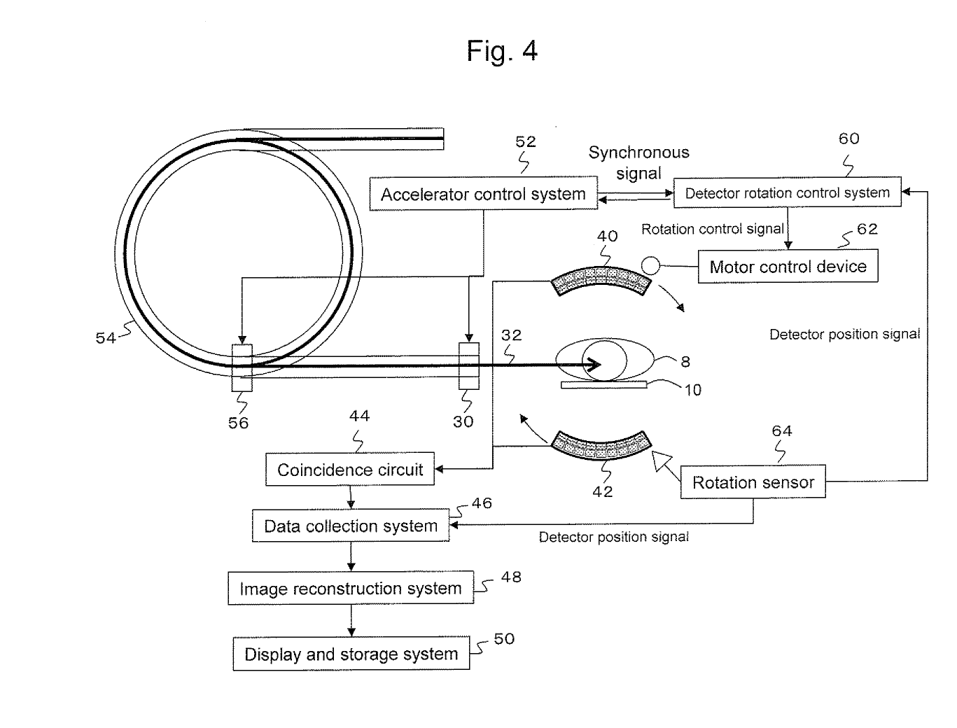

FIG. 4 shows a mechanism for synchronizing beam irradiation with the rotation of the detectors 40 and 42. The irradiation period of the treatment beam 32 is controlled by an accelerator control system 52. A synchrotron 54 basically makes intermittent operations to repeat beam irradiation ON and OFF. A technology for producing a beam from a synchrotron continuously is also under development. In the diagram, 56 designates a beam outlet part.

A detector rotation control system 60 transmits a rotation control signal to a motor control unit 62 so that the rotation of the detectors 40 and 42 is synchronized with a synchronous signal received from the accelerator control system 52. Information on the position and rotation speed of the detectors 40 and 42 is successively transmitted from a rotation sensor 64 to the detector rotation control system 60.

Single event data on annihilation radiations detected by detectors such as PET detectors is converted by a coincidence circuit 44 into coincidence data for identifying lines of coincidence. The coincidence data is stored into a data collection system 46 in succession. After accumulation of measurement data for a certain period of time, an image reconstruction system 48 performs an image reconstruction operation, and displays or stores the images of the irradiation field. The time width for accumulating measurement data will be referred to as a time frame. The processing systems of the PET measurement data may basically continue processing and collecting measurement data independent of the accelerator control system 52 and the shield control system 60. It is needed, however, to include a detector position signal or the like into the coincidence data so that the absolute positions of the lines of coincidence can be identified.

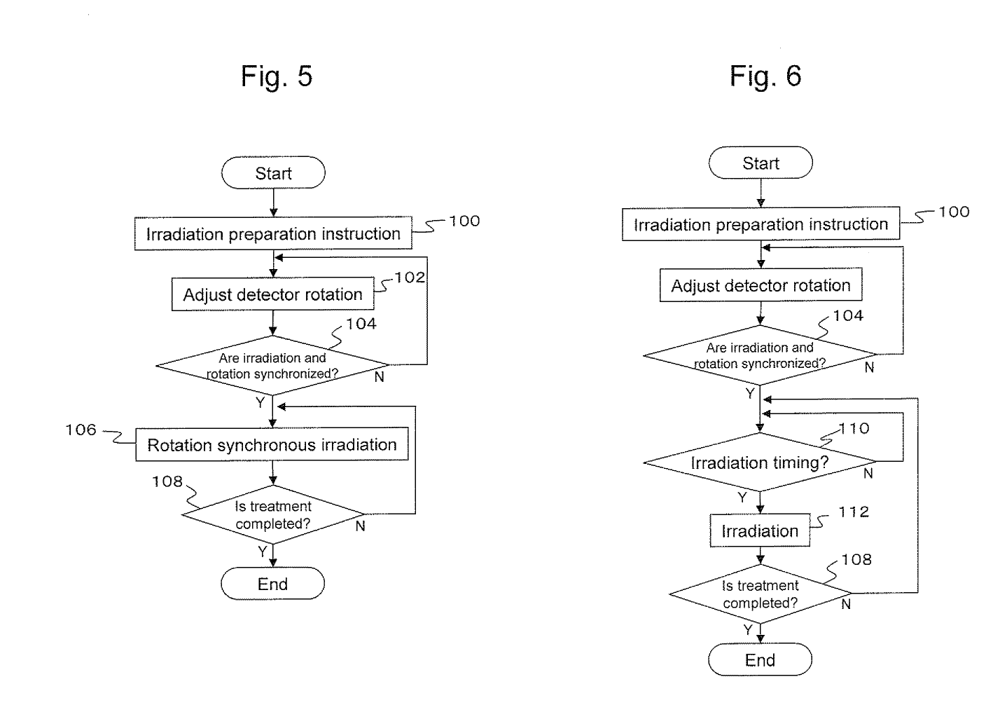

FIG. 5 shows a typical procedure of beam irradiation in synchronization with detector rotation. The detector rotation control system 60 acquires an irradiation preparation instruction (step 100), and makes adjustments so as to synchronize the operation period of the synchrotron with the detector rotation (step 102). Specifically, their periods and phases are synchronized. If the irradiation and the detector rotation are synchronized (step 104), rotation synchronous irradiation (step 106) of performing irradiation only when no detector lies in the critical region is repeated until the treatment is completed (step 108). The foregoing has dealt with the control of synchronizing the detector rotation with the operating period of the synchrotron. When continuously producing a beam from the synchrotron, the beam outlet part 56 may be controlled so that the irradiation timing matches the detector rotation after the detector rotation is stabilized.

FIG. 5 shows the procedure in which the accelerator control system 52 performs the control for rotation synchronous irradiation on the assumption that the irradiation and rotation are stably in synchronization with each other. When, for example, the detector rotation is unstable, as shown in FIG. 6, the detector rotation control system 60 may check the detector position and transmit irradiation timing information to the accelerator control system 52 (steps 110 and 112).

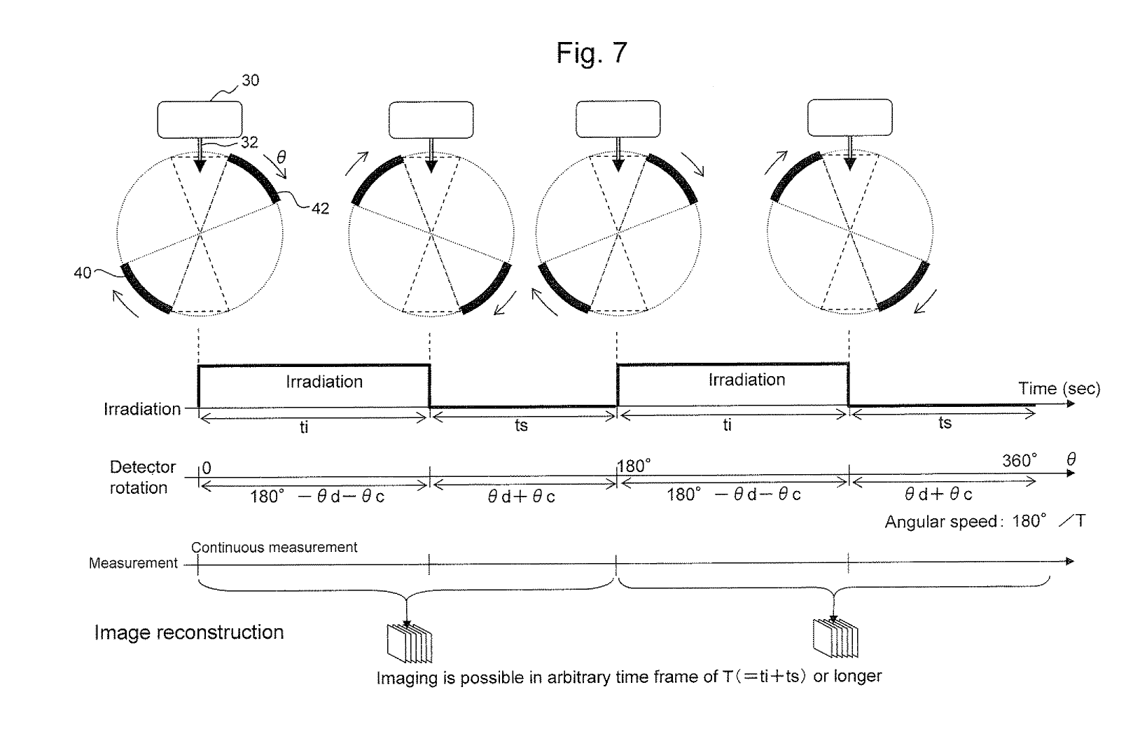

FIG. 7 shows how beam irradiation and detector rotation are synchronized to prevent the detectors from interfering with a treatment beam or being affected by nuclear fragments. The treatment beam irradiation is performed only when no detector is in the critical region. The treatment beam irradiation is turned OFF when a detector approaches the critical region. The treatment beam is operated in periods of T=ti+ts sec, with ti sec of irradiation followed by ts sec on standby. The PET device makes a single rotation in 2 T sec. The condition on θd, a parameter related to detector size, will be described below. The lower limit of θd is:

θd≧2 sin⁻¹(r/R),

where R is the radius of the orbit of the detectors, and r is the radius of the PET field of view. The upper limit of θd is:

θd≦ts/T×180°−θc.

If a beam can be continuously produced from the synchrotron, the beam outlet part 56 may be controlled so that the irradiation timing matches the detector rotation, with ti sec of irradiation followed by ts sec on standby. For spot scanning irradiation, the time to switch range shifters may be assigned to the standby time of ts sec. If the irradiation time ti and the standby time ts vary, the detector rotation speed may be adjusted accordingly. If the irradiation pattern is such that a series of irradiations continues, or if the standby time ts is extremely short, a duration equivalent to the series of irradiations may be collectively assigned to the irradiation time ti.

For PET measurement, the collection of coincidence data is continued. Data as much as a time frame specified afterward is extracted for image reconstruction. Alternatively, a time frame may be specified in advance, and PET measurement may be performed only for the time frame specified. In any case, lines of coincidence from various angles are needed for image reconstruction. The minimum value of the time frame capable of imaging the irradiation field is an irradiation clock of T sec which is equivalent to a 180° rotation of the PET detectors. If the number of counts of annihilation radiation measured is small, a time frame longer than T sec may be set to improve the S/N ratio of the measurement data. It is known that prompt gamma rays are also emitted from the irradiation field during irradiation aside from annihilation radiations. Prompt gamma rays can increase random coincidence which is a noise component to PET measurement. In view of ON/OFF periodicity of microsecond order during ti sec of irradiation, countermeasures have been proposed that use only measurement data in OFF states for image reconstruction, excluding measurement data in ON states. (P. Crespo, et al., “Suppression of random coincidences during in-beam PET measurements at ion beam radiotherapy facilities,” IEEE TRANSACTIONS ON NUCLEAR SCIENCE, VOL. 52, NO. 4, AUGUST 2005).

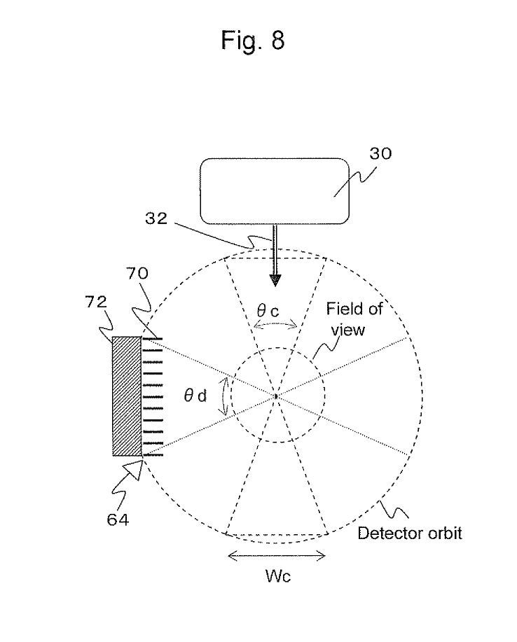

The imaging device need not necessarily be a PET device, and may be a SPECT device with a gamma camera as shown in FIG. 8 or the like. In such a case, it would be possible to measure the aforementioned prompt gamma rays as a signal aside from annihilation radiations. In the diagram, 70 designates a collimator, and 72 designates a detector.

### EXAMPLES

The Heavy Ion Medical Accelerator in Chiba (HIMAC) of the National Institute of Radiological Sciences in Japan performs treatment beam control with a period of T=3.3 sec. The present invention will be described in terms of application to HIMAC. Assuming that the radius of the orbit of the detectors R=50 cm and the radius of the PET field of view r=20 cm, the lower limit of θd is θd≧47.2°. Table 1 shows upper limits of θd for different irradiation durations ti and different widths We of the critical region. ts=3.3−ti. No device is feasible if the upper limit falls below the lower limit (in the table, denoted as NA). For enhanced sensitivity of the PET device, it is actually desirable to employ the maximum value of θd.

TABLE 1

| | Wc = | Wc = | Wc = | Wc = | Wc = |
|---|---|---|---|---|---|
| | 20 cm | 30 cm | 40 cm | 50 cm | 60 cm |
| ti = 0.5 sec | 129.7° | 117.8° | 105.6° | 92.7° | 79.0° |
| ti = 1.0 sec | 102.4° | 90.5° | 78.3° | 65.5° | 51.7° |
| ti = 1.5 sec | 75.1° | 63.3° | 51.0° | NA | NA |
| ti = 2.0 sec | 47.8° | NA | NA | NA | NA |

While the example has dealt with the case with a single irradiation port, the present invention is also applicable with a plurality of irradiation ports such as vertical one and horizontal one. Typically, a plurality of irradiation ports are not simultaneously used for treatment beam irradiation. The phase of PET rotation or the phase of beam irradiation may therefore be relatively changed depending on the movement of the port for beam irradiation. The same holds for a rotating irradiation gantry.

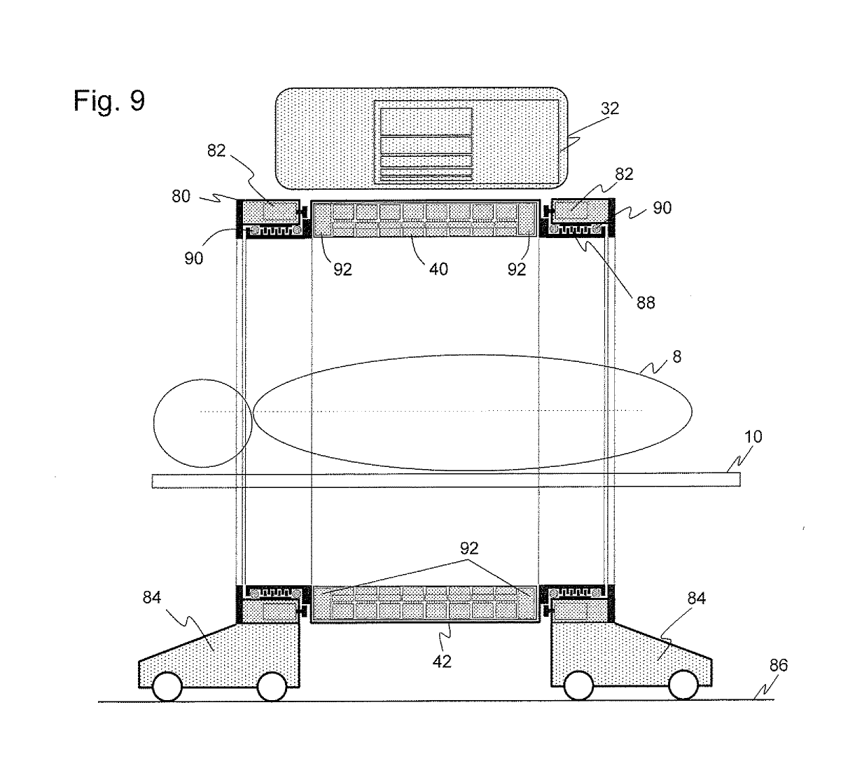

FIG. 9 shows a practical example of the detector rotation type radiation therapy and PET hybrid device according to the present invention. PET detectors 40 and 42 to be rotated by a rotating motor 82 are sandwiched between support rings 80 at both ends. Carriage portions 84 of the support rings are fixed onto rails 86 which are installed on the floor of a therapy room. Power supply and signal transmission between the rotating PET detectors 40 and 42 and the support rings 80 are conducted through slip rings 88. In the diagram, 90 designates ball bearings, and 92 designates front end circuits.

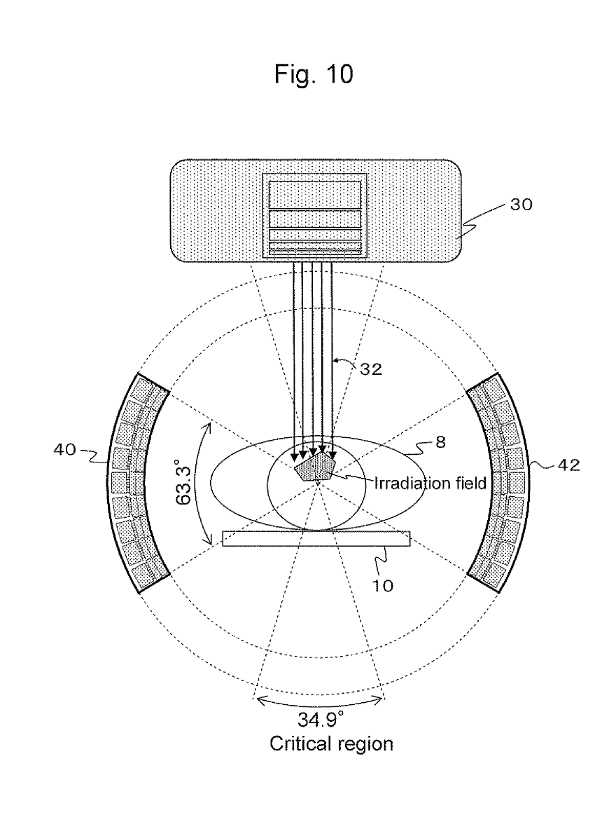

FIG. 10 is a cross-sectional view of the example of FIG. 9 near the center. In the example, the detector size of θd=63.3° is employed, which is the maximum allowable value at Wc=30 cm (θc=34.9°) and ti=1.5 sec.

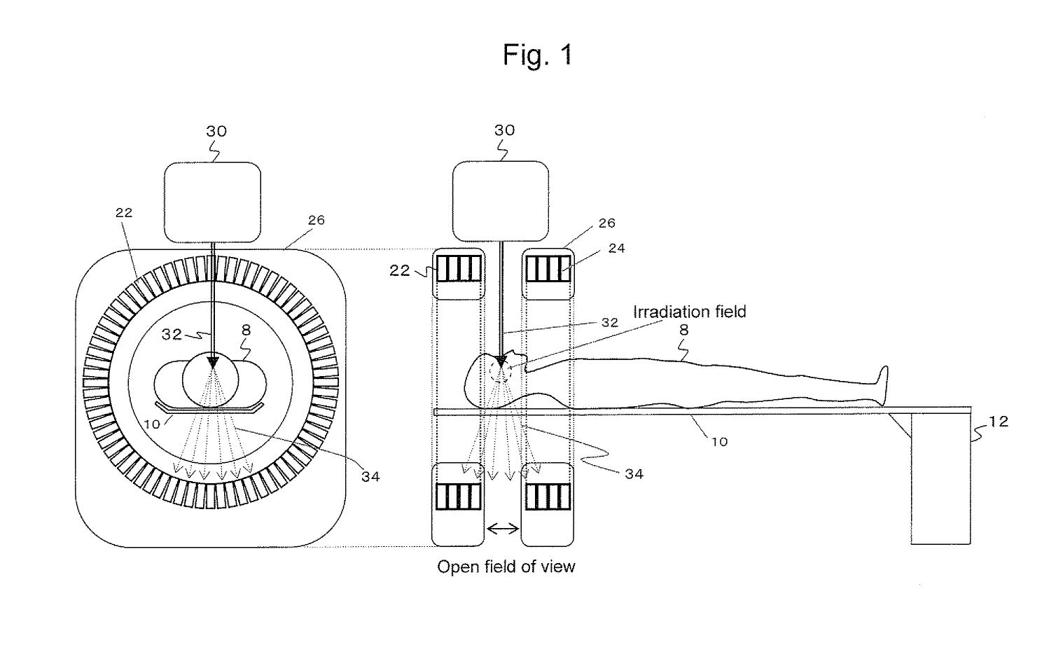

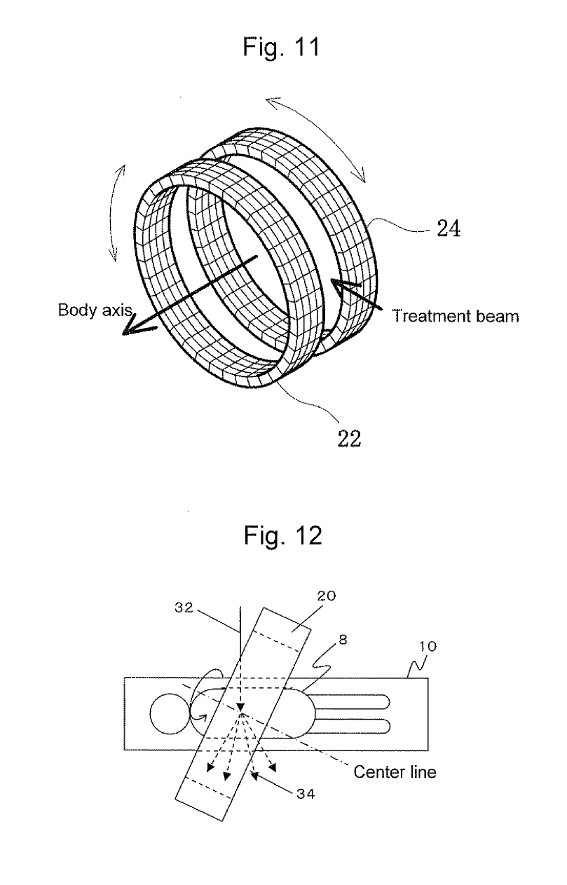

FIG. 11 shows an example where the PET detector rotation according to the present invention is applied to an open PET device. In an open PET device, the treatment beam can be introduced into the irradiation field through an open space without interfering with detectors. As shown in FIG. 1, attention needs to be paid to nuclear fragments incident on the detectors. Nuclear fragments are generated with forward directivity with respect to the treatment beam. Consequently, among the detectors 22 and 24 of ring-shaped arrangement, detectors lying on the side close to the irradiation port and ones lying on the opposing side undergo intensive incidence of nuclear fragments. The incidence of nuclear fragments on the detectors on the side close to the irradiation port can be suppressed by the insertion of a shielding material between the irradiation port and the detectors, like the inclusion of shielding material into gantry members. Such a shielding material, however, can reduce not only nuclear fragments but annihilation radiations to be measured as well. It is therefore undesirable to install a shielding material in front of the detectors lying on the opposing side. Then, the detector rings 22 and 24 can be rotated to distribute and lower the degree of radioactivation of the PET detectors due to the incidence of nuclear fragments.

Specifically, the detector rings 22 and 24 are rotated at least during irradiation. The detector rings may be continuously rotated. Since no high-speed rotation such as described in the previous example is needed, ±180° reciprocating rotations are preferred since wiring without slip rings can simplify the device. Rotations may be either continuous or intermittent step by step. The speed may be either constant or variable. The two rings need not necessarily have the same rotation speed or direction. The present method is characterized by having no limitation on how to produce a treatment beam from the accelerator. The treatment beam may be emitted continuously. If the irradiation of the treatment beam is performed in cycles of T sec, it is preferred that the rotation period is not an integer multiple of T so that the incidence of nuclear fragments will not concentrate on some detectors.

The rotation may be stopped during irradiation. The degree of radioactivation of the PET detectors due to the incidence of nuclear fragments may be sensed and the detector rings may be rotated to change the positions of detectors on which nuclear fragments are incident, so as to make damage accumulation uniform.

According to the method shown in FIG. 11, there are no missing detectors in the angular directions. Lines of coincidence from any angle needed for image reconstruction can thus be measured constantly. Unlike a method of rotating opposed gamma camera type, there is a characteristic that the irradiation field can be imaged in an arbitrary time frame.

The method of rotating the PET detectors at least during irradiation or sensing the degree of radioactivation and rotating the detectors according to the present invention may be applied to devices other than an open PET device. FIG. 12 is a prior example where an ordinary PET detector ring 20 is obliquely arranged (P. Crespo, at al., “On the detector arrangement for in-beam PET for hadron therapy monitoring,” Phys. Med. Biol., vol. 51 (2006), pp. 2143-2163). While there is provided a beam irradiation path, nuclear fragments 34 can be incident on detectors as shown in the diagram. In contrast, it is possible to distribute the incidence of nuclear fragments 34 to reduce damage to the detectors by rotating the detector ring 20 about its center line as shown by the arrow in the diagram at least during irradiation, or by sensing the degree of radioactivation and rotating the detector ring 20.

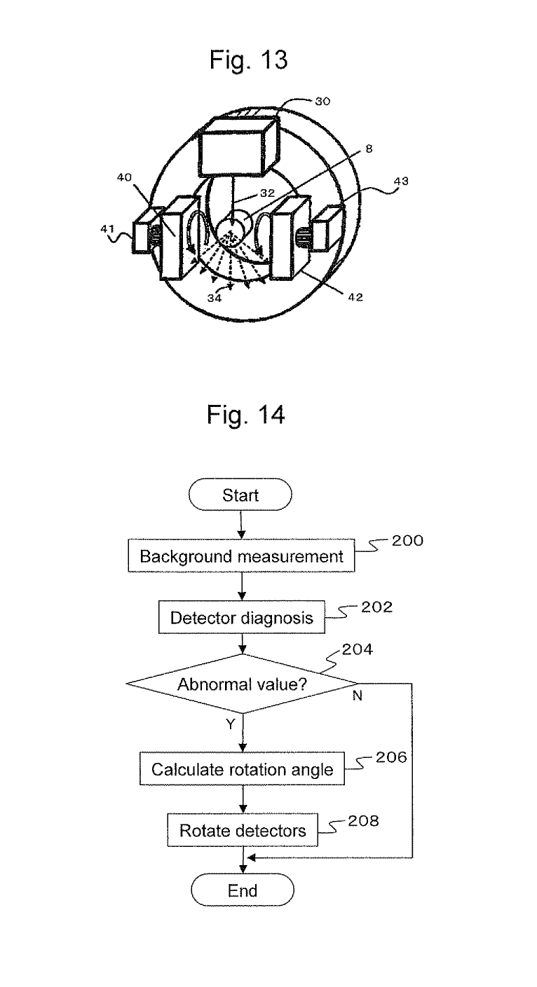

FIG. 13 shows an example where the present invention is applied to a prior example of an opposed gamma camera type PET device (Japanese Patent Application Laid-Open No. 2008-022994, Japanese Patent Application Laid-Open No, 2008-173299). For improved device sensitivity, PET detectors 40 and 42 need to be increased in size. This may lead to the incidence of nuclear fragments 34 on the bottom ends of the PET devices 40 and 42. In such a case, it is possible to distribute the incidence of nuclear fragments 34 to reduce damage to the detectors by sensing the degree of radioactivation and rotating the PET detectors 40 and 42 by 90° or 180° by rotation drive devices 41 and 43 as shown by the arrows in the diagram.

FIG. 14 shows a typical procedure for sensing the degree of radioactivation and rotating detectors to distribute the incidence of fragments for reduced detector damage. The sensing of the degree of radioactivation of the detectors is characterized in that it can be performed by using a part of the functions of an ordinary PET measurement system without the provision of special detection devices. Specifically, background radiation measurement is initially performed (step 200) with no patient in the field of view nor irradiation, i.e., without any radiation source in the field of view. The measurement may be coincidence measurement. For efficient measurement of radiations other than annihilation radiations, i.e., so-called single photon emission, single event data is desirably accumulated before coincidence counting. The measurement is continued for a certain period of time before measurements per unit time are calculated for each detector element, such as in units of blocks. In detector diagnosis (step 202), detector elements whose measurements exceed a predetermined value are determined to be abnormal (step 204). The angle of detector rotation is then calculated so as to keep abnormal detector elements away from the position of incidence of nuclear fragments (step 206), and the detectors are rotated (step 208).

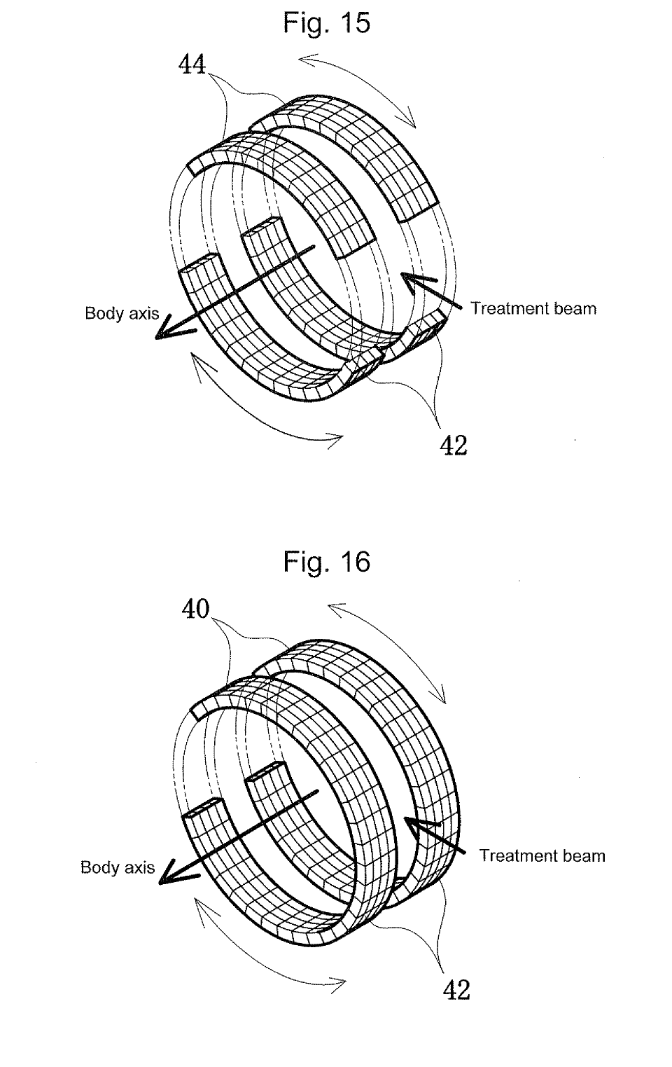

FIG. 15 is another example where the method of rotation type PET according to the present invention is applied to an open PET device. Two devices shown in FIGS. 9 and 10 are arranged apart in the direction of the body axis of the patient, and control is performed to match the rotation phases of the two PET devices. Alternatively, some detectors at the centers of the PET detectors 40 and 42 shown in FIGS. 9 and 10 may be removed, i.e., as if two rotating PET devices are physically coupled to each other.

An open PET device by definition allows introduction of a treatment beam into the irradiation field through an open space without interference with the detectors. The problem of the incidence of nuclear fragments on detectors on the side close to the irradiation port can be suppressed by the insertion of a shielding material between the irradiation port and the detectors, like the inclusion of shielding material into gantry members. Consequently, while FIG. 15 shows the configuration in which both sides of the detector rings are removed, the detector rings have only to be cut in one side alone. FIG. 16 shows a configuration in which unnecessary gaps are filled with detectors to improve the sensitivity of PET measurement.

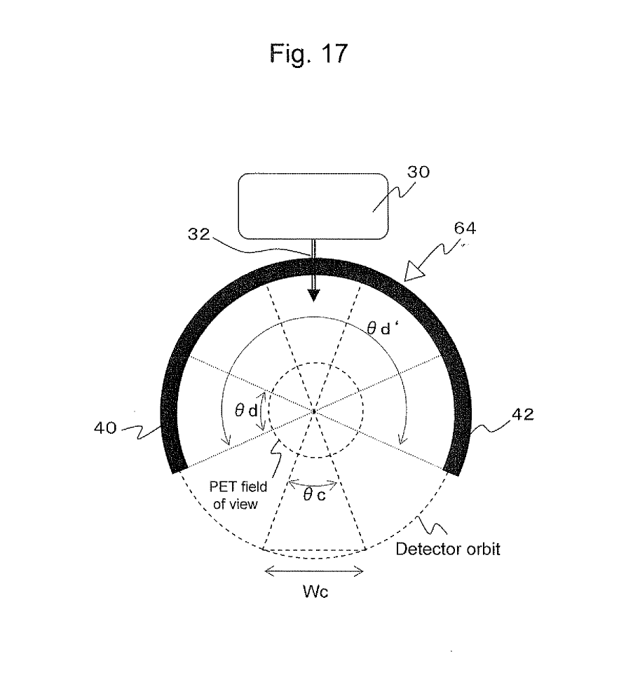

FIG. 17 shows the configuration of the PET device shown in FIG. 16. The PET device has the structure that arc-shaped PET detectors are arranged with an angle of view θd′ when seen from the center of rotation of the detectors. θd represents the range where PET detectors lie symmetrically with respect to the center of rotation of the detectors. θd satisfies the relationship θd′=θd+180°.

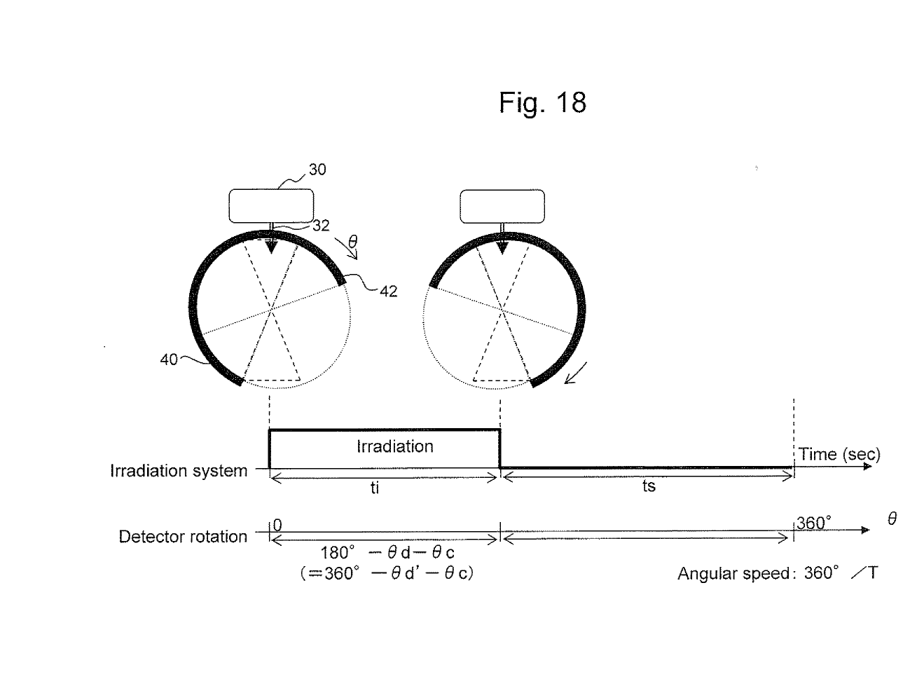

FIG. 18 shows how beam irradiation and detector rotation are synchronized to avoid the incidence of nuclear fragments on the detectors. The treatment beam irradiation is performed only when no detector is in the critical region. The treatment beam irradiation is turned OFF when the detectors approach the critical region. The treatment beam is operated in periods of T=ti+ts sec, with ti sec of irradiation followed by ts sec on standby. While in FIG. 7 the PET device makes a rotation in 2 T sec, the present mode with a single location of missing detectors needs to make a rotation in T sec. As in FIG. 7, the lower limit of θd is:

θd≧2 sin⁻¹(r/R),

where R is the radius of the orbit of the detectors, and r is the radius of the PET field of view. The upper limit of θd is:

θd≦180°−ti/T×360°−θc.

Table 2 shows upper limits of θd for different irradiation durations ti and different widths Wc of the critical area under the same condition of the beam irradiation period T=3.3 sec, the radius of the orbit of the detectors R=50 cm, and the radius of the PET field of view r=20 cm. The lower limit of θd is θd≧47.2°. No device is feasible if the upper limit falls below the lower limit (in the table, denoted as NA). For enhanced sensitivity of the PET device, it is actually desirable to employ the maximum value of θd. The angle of view θd′ from the center of rotation of the detectors is given by θd÷=θd+180°. As compared to the case of Table 1, given the same beam irradiation period T, shorter irradiation durations ti are needed since the rotation speed of the PET detectors is twice as high.

TABLE 2

| | Wc = | Wc = | Wc = | Wc = | Wc = |
|---|---|---|---|---|---|
| | 20 cm | 30 cm | 40 cm | 50 cm | 60 cm |
| ti = 0.5 sec | 102.3° | 90.5° | 78.3° | 65.5° | 51.7° |
| ti = 1.0 sec | 47.8° | NA | NA | NA | NA |
| ti = 1.5 sec | NA | NA | NA | NA | NA |
| ti = 2.0 sec | NA | NA | NA | NA | NA |

### INDUSTRIAL APPLICABILITY

When performing monitoring for detecting annihilation radiations occurring from an irradiation field due to radiation irradiation in radiation therapy which is conducted by irradiating an affected area with X-rays or a particle beam, it is possible to reduce the incidence of nuclear fragments on detectors without interfering with the treatment beam, thereby enabling measurement of annihilation radiations and three-dimensional imaging of the irradiation field immediately after irradiation or even during irradiation.

1. A detector rotation type radiation therapy and imaging hybrid device comprising an imaging device that has a detector arranged so as to be able to measure a secondary radiation occurring from an affected area due to radiation irradiation and images an irradiation field after irradiation or during irradiation in synchronization with a radiation with which a field of view of the detector is irradiated,
the hybrid device comprising:
a radiation therapy device that irradiates a part of a subject with a radiation from a predetermined direction, the part lying in a field of view of the imaging device;
the detector that is arranged so as to be rotatable about the field of view; and
means for controlling rotation of the detector so as to lessen incidence of nuclear fragments on the detector, the nuclear fragments flying forward in a direction of irradiation from the subject due to the radiation irradiation.

2. The detector rotation type radiation therapy and imaging hybrid device according to claim 1, wherein the detector is a group of detectors that are opposed to and paired with each other with the subject interposed therebetween so as to be capable of coincidence measurement of a pair of annihilation radiations occurring from the subject, and the imaging apparatus is a PET device that performs tomographic scanning on the subject.

3. The detector rotation type radiation therapy and imaging hybrid device according to claim 1, wherein the irradiation of the radiation is performed in a region where nuclear fragments are not incident on the detector, and the irradiation of the radiation is stopped when the detector approaches a region where nuclear fragments are incident, the regions lying on an orbit of rotation of the detector.

4. The detector rotation type radiation therapy and imaging hybrid device according to claim 1, wherein the group of detectors is formed in a discontinuous ring shape; a radiation irradiation path for irradiating the subject with the radiation is arranged to pass the discontinuous ring; and rotation of the group of detectors is controlled to locate a discontinuous part of the ring to a position across the radiation irradiation path during radiation irradiation so that the subject is irradiated with the radiation through the discontinuous part.

5. The detector rotation type radiation therapy and imaging hybrid device according to claim 4, wherein a plurality of the discontinuous part are disposed, and the discontinuous part to be located across the radiation irradiation path is switched based on a predetermined plan when radiation irradiation is at rest.

6. The detector rotation type radiation therapy and imaging hybrid device according to claim 3, wherein the group of detectors is formed in a ring shape about an axis of the subject; two of the ring-shaped detector are opposed to each other with a gap therebetween; a radiation irradiation path for irradiating the subject with the irradiation is arranged in the gap; and a detector on the ring of the ring-shaped detector is missing.

7. The detector rotation type radiation therapy and imaging hybrid device according to claim 6, wherein opposing two detectors on the ring of the ring-shaped detector are missing.

8. The detector rotation type radiation therapy and imaging hybrid device according to claim 1, wherein the detector is formed in a ring shape, and the ring-shaped detector makes continuous rotations during radiation irradiation to distribute a degree of radioactivation over respective detectors.

9. The detector rotation type radiation therapy and imaging hybrid device according to claim 8, wherein, when the radiation irradiation is periodically performed, a period of rotation of the ring-shaped detector is other than an integer multiple of a period of irradiation of the radiation.

10. The detector rotation type radiation therapy and imaging hybrid device according to claim 1, wherein a degree of radioactivation of the detector radioactivated by the nuclear fragments is detected, and if the degree of radioactivation of a detected part is detected to reach or exceed a predetermined value, the ring-shaped detector is rotated and retracted by a predetermined angle to a position of less radioactivation.

11. The detector rotation type radiation therapy and imaging hybrid device according to claim 10, wherein the predetermined angle is an angle set in advance.

12. The detector rotation type radiation therapy and imaging hybrid device according to claim 10, wherein the predetermined angle is an angle by which the ring-shaped detector is rotated after the detecting means detects that the degree of radioactivation of the detected part reaches or exceeds a first predetermined value, until a level of radioactivation detected by the detecting means falls to or below a second predetermined value that is a level lower than or equal to the first predetermined value.

13. The detector rotation type radiation therapy and imaging hybrid device according to claim 8, wherein two of the ring-shaped detector are opposed to each other with a gap therebetween, a radiation irradiation path for irradiating the subject with the radiation is arranged in the gap.

14. The detector rotation type radiation therapy and imaging hybrid device according to claim 8, wherein an axis of the ring-shaped detector is oblique to an axis of the subject.

15. The detector rotation type radiation therapy and imaging hybrid device according to claim 8, wherein the group of detectors is opposed to beside the subject.

16. The detector rotation type radiation therapy and imaging hybrid device according to claim 10, wherein the degree of radioactivation of the detector radioactivated by the nuclear fragments is detected, and if the group of detectors formed in the ring shape has a plurality of discontinuous parts and the degree of radioactivation of the detected part is detected to reach or exceed a predetermined value, the discontinuous part to be located across the radiation irradiation path is switched when the radiation irradiation is at rest.

17. The detector rotation type radiation therapy and imaging hybrid device according to claim 8, wherein the degree of radioactivation is detected from a measured value per unit time, the measured value being calculated for each element of the detector.

18. The detector rotation type radiation therapy and imaging hybrid device according to claim 1, wherein the group of detectors make a swing movement.

19. The detector rotation type radiation therapy and imaging hybrid device according to claim 18, wherein a swing angle is smaller than or equal to 360°.

20. A control program of a detector rotation type radiation therapy and imaging hybrid device including an imaging device that has a detector arranged so as to be rotatable about a subject and so as to be able to measure a radiation occurring from an affected area due to radiation irradiation and images an irradiation field after irradiation or during irradiation in synchronization with a radiation with which a field of view of the detector is irradiated,
the control program controlling rotation of the detector so as to lessen incidence of nuclear fragments on the detector, the nuclear fragments flying forward in a direction of irradiation from the subject due to the radiation irradiation.

21. The control program of a detector rotation type radiation therapy and imaging hybrid device according to claim 20, wherein the detector is a group of detectors that are opposed to and paired with each other with the subject interposed therebetween so as to be capable of coincidence measurement of a pair of annihilation radiations occurring from the subject, and the imaging apparatus is a PET device that performs tomographic scanning on the subject.

22. The control program of a detector rotation type radiation therapy and imaging hybrid device according to claim 21, wherein if a degree of radioactivation of the detector radioactivated by the nuclear fragments is detected to reach or exceed a predetermined value, the group of detectors is rotated by an angle set in advance.

23. The control program of a detector rotation type radiation therapy and imaging hybrid device according to claim 21, wherein if a degree of radioactivation of the detector radioactivated by the nuclear fragments is detected to reach or exceed a first predetermined value, a ring-shaped detector is rotated until a level of radioactivation detected falls to or below a second predetermined value that is a level lower than or equal to the first predetermined value.

24. The control program of a detector rotation type radiation therapy and imaging hybrid device according to claim 21, wherein the group of detectors make a swing movement with a switch angle of 360° or less.
